(19) United States
(12) Patent Application Publication
(10) Pub. No.: US 2003/0143593 A1
(43) Pub. Date: Jul. 31, 2003

(54) Quantative method for measuring gene expression

(76) Inventors: Fabienne Hermitte, La Ciotat (FR); Vincent Fert, Allauch (FR)

Correspondence Address: YOUNG & THOMPSON, 745 SOUTH 23RD STREET 2ND FLOOR, ARLINGTON, VA, 22202

(21) Appl. No.: 10/281,255

(22) Filed: Oct. 28, 2002

Related U.S. Application Data
(63) Continuation of application No. PCT/FR01/01293, filed on Apr. 26, 2001.

(30) Foreign Application Priority Data
Apr. 26, 2000 (FR) 00/05322

(51) Int. Cl. C12Q 1/68; C07H 21/04; C12P 19/34

(52) U.S. Cl. 435/6; 435/91.2; 536/24.3

(57) ABSTRACT

The invention concerns a method for quantitative measuring of gene expression by obtaining marked probes of pre-selected homogeneous size, after reverse transcription and amplification. The invention also concerns the method for preparing marked probes, and implementing kits using microarrays and macroarrays.

The present invention relates to a process for carrying out quantitative measurements of the level of expression or variability of expression in large assemblies of genes using the DNA chip technique.

The terms used throughout the text have the following meanings:

PROBE: means any sequence of labeled nucleic acids representative of a complex mixture that is to be studied and the elements of which are to be analyzed; said probes are obtained by transcription and/or reverse transcription of the starting complex nucleic acid mixture, if necessary followed by a sequence amplification method;

Said probes are directly or indirectly labeled to emit a detectable signal using conventional techniques. Labelling can be radioactive labelling, notably with phosphorus ³²P or phosphorus ³³P, or non-isotopic, such as enzymatic or fluorescent labelling. Single strand DNA, double strand DNA or RNA can be concerned.

TARGET: the term “target” means nucleic acid sequences fixed in an ordered manner on a chip. Said sequences are generally known; they may be a single strand nucleic acid or a double strand nucleic acid. They can be hybridized with probes representing the population to be studied.

CALIBRATION: means a process for producing a population of nucleic acids representative of a starting population and which is substantially homogeneous as regards size. The term “substantially homogeneous” means that for a given size, the difference in length does not exceed 20%, and preferably 10%, for lengths longer than the selected size, and 50% for lengths that are below the selected size.

CHIP: as used here means any support that carries an ordered chip of target nucleotide or. oligonucleotide sequences that can be hybridized with probes. The supports can be produced from silica or glass or an organic polymer type nylon or nitrocellulose. The nucleotide or oligonucleotide sequences can be fixed in position by any means known to the skilled person from the moment at which hybridization with the target sequences is possible. The terms “chip”, “microarray”, “macroarray” will be employed here. Reference should be made advantageously to S. GRANJEAUD et al., (1) for the characteristics of the different chip technologies.

TRANSCRIPTOME: the term “transcriptome” means the totality of RNA extracted or purified from a cell or a cell population. A transcriptome reflects genome expression. The relative quantities of each RNA reflect expression of the corresponding gene; a modification in this rate of expression can result either from a modification in the expression of the corresponding gene or from a modification in the stability of the RNA in question. In any case, this modification may have consequences on the structure and/or quantity of synthesized proteins in the case of messenger RNA.

NUCLEIC ACID: the term “nucleic acid” as used here means any single strand/double strand sequence expressed or copied from the moment at which is it hybridisable with messenger RNA of the transcriptome or its complementary sequence.

Identification of genetic expression in response to different physiological or pathological conditions appears to be an essential approach in elucidating the molecular mechanisms associated with certain pathologies, therapeutic treatments or with a particular state in the organs or tissues development.

Chip technology, on which high density of cDNA or ESTs are fixed, offers a direct approach for this type of analysis.

Large-scale measurements of expression are all based on the use of a complex probe prepared from a transcriptome and hybridized with an array of several hundred or thousand targets each representing a different gene. The great strength of this approach is its parallelism: each hybridization provides information on each gene represented in the array and the information is cumulative. There are two obvious reasons to explain the increasing interest shown in such techniques: the first is that knowing the level of expression of a gene in different tissues or under different physiological or pathological situations enables hypotheses to be formulated regarding its role; the second is technical in nature: said data is currently the only data that can be obtained for large assemblies of genes, and they are in keeping with the continuity of cDNA libraries or whole genomes sequencing.

The joint study of a large number of sequences and tissues in which the corresponding genes are expressed and comparing the profiles obtained from healthy and diseased tissue or in different situations in the same tissue enables target genes to be identified which may be involved in a biological phenomenon. This is termed “gene discovery”. It is also possible to choose to work with a smaller number of known genes that have been selected a priori for their certain or supposed involvement in a biological process. The measurement of the expression profile noted on this array of genes is then used to characterize the state of a studied tissue and to obtain diagnostic indications or prognostic elements.

Within the context of large-scale expression studies (on DNA chips), complex probes have been produced from a mixture of RNA (total or messenger RNA, extracted for example from a tissue or from a cell culture). Those probes are then hybridized with target DNA sequences deposited on a support (Nylon membrane, glass . . . ). Quantification of the signals obtained for each target enable the quantity of corresponding RNA in the tissue (or cell culture) being studied to be determined. Producing the complex probe is a key step as the products present in the probe (products obtained after reverse transcription of mRNA) must rigorously reflect the representation of each mRNA in the initial mixture. The efficacy of the reverse transcription reaction is not the same for all RNA sequences: very stable secondary structures can, for example, block the cDNA synthesis reaction. Most of the time, labeled nucleotides are incorporated at this step to allow detection of hybridization signals. The measurement obtained for two messenger RNA present in equal concentrations can be different because of two distinct. phenomena: i) if the two RNA differ in size; or ii) for identically sized RNA, if reverse transcription in one of the two is stopped prematurely, producing two cDNA with different sizes. Those two phenomena introduce the same bias: during labelling, the longest fragment incorporates more labeled nucleotides and its measurement is overestimated. Under conventional reverse transcription conditions, the size of the cDNA molecules obtained is in the range 7 kb to 300 nucleotides, as demonstrated in the publication by Rajeevan MS et al., (5). The authors of the publication by Chen J J W et al., (1998) (4) emphasizes the problem of bias in the measurements due to size differences of the probe transcripts.

Thus, there is a genuine need to find a reliable method that can transcribe the complex population of mRNA into cDNA of homogeneous size so that the cDNA all have the same specific activity, and the cDNA mixture obtained has the exact representation (in terms of molecules number) of the initial mRNA mixture. Hybridization of the probe on a DNA chip will then accurately reflect the RNA representation in the initial mixture and will generate reproducible data which are perfectly quantitative, allowing not only expression differentials to be produced (for example for different physiological conditions), but also allowing accurate measurements of the number of transcripts to be produced for a given condition.

In the same manner, large-scale expression studies usually require large quantities of RNA (conventionally derived from a few milligrams of tissue or from a few million cells), to produce complex probes that can reliably detect less abundant messenger RNA. This constraint means that a step has to be carried out for amplifying RNA or corresponding cDNA for application to the study of tissues or cells available only in very small quantities, as is the case in certain biopsies, for example. Thus, there is a genuine need for a reliable method that can, from a small number of cells (and thus small quantities of RNA) homogeneously amplify the complex population of mRNA to produce a probe in a sufficient quantity for hybridization of DNA chips, which probe reflects the RNA representation in the initial mixture (both for strongly expressed RNA and for weakly expressed RNA) and which generates reproducible hybridization data.

Different amplification methods have already been applied to producing complex probes. The most frequently used method is amplification by PCR. This technique can amplify a DNA sequence, but only if the 5′ and 3′ ends sequences are known, to allow the primers either side of the sequence to be amplified to be selected. With a complex mixture of messenger RNA in which the sequences are unknown, these primers can be introduced during steps for synthesizing the first strands (by reverse transcription) and second strands, and we then speak of anchored PCR. Different methods of anchored PCR have been previously described; the most frequently used method is to initiate the reverse transcription step with a poly T oligonucleotide flanked by a known sequence (3′ anchor) then add a homopolymeric tail (for example Gs) to the 3′ end of the newly synthesized ss cDNA (single strand complementary DNA) thanks to the enzymatic activity of the terminal deoxyribonucleotide transferase enzyme (TdT). The second strand is then synthesized from a poly C primer (deoxycitidines) flanked by a known sequence (5′ anchor). The double strand DNA obtained are then amplified by nested PCR from primers selected from the 5′ and 3′ anchors. The problem with PCR amplification is that the amplification yield of a nucleotide fragment is a function of the size of concerned fragment: a short fragment co-amplified by PCR with a longer fragment will always be preponderant in the final reaction product, even if it was less abundant in the initial mixture. This bias becomes particularly annoying when exactly the expression level of the genes is to be studied: it is absolutely essential that each mRNA is present in the same proportions in the complex probe as in the cell type or tissue from which it derives.

Actually, for each mRNA present in the initial mixture, the size of amplified product will depend on the one hand, of efficacy of the reverse transcription step and on the other hand, of initiation of synthesis of the second strand and amplification steps. During the amplification steps using PCR, non specific annealing inside one of the two strands is sufficient for the non specific product (shorter than the expected specific fragments) to go into majority in the final reaction product. For small sized products, the amount of radioactivity, fluorescence, or reporter molecule (for example biotin) incorporated per molecule will be less important than for the larger products, again introducing a potential difference in intensity of signals obtained after hybridization of chips.

A further RNA amplification technique, this time a linear technique, has also been described (Van Gelder et al., 1990, PNAS 87: 1663-1667). Using reverse transcription of messenger RNA initiated from an oligonucleotide containing a plurality of Ts (complementary to the poly A tail of the messenger RNA) flanked by a sequence recognized by the RNA polymerase of the T7 phage, this method can produce single strand cDNA comprising the promoter sequence for T7 RNA Pol genome at their 5′ end. Synthesis of a complementary double strand of the single strand cDNA is then initiated using different protocols:

either by the method known as the hairpin loop method (Maniatis et al., 1978, Cell, 15: 687-701) which involves a reaction with S1 nuclease which is difficult to control and results in rearrangements in the portion corresponding to the 5′ ends of the mRNA;

or using the method known as the Gubler and Hoffman method (Gubler and Hoffman, 1983, Genet., 25: 263-269) which can also generate hairpin loops and require treatment with S1 nuclease;

or using a tailing technique, which consists of adding, thanks to the enzymatic activity of TdT, a homopolymeric tail to the 3′ end of neo-synthesized single strand cDNA then initiating synthesis of second strand from a complementary oligonucleotide of the homopolymeric tail.

The double strand products obtained are then amplified by incubation with the enzyme T7 RNA polymerase. However, for each mRNA present in the initial mixture, the size of amplified product will depend on the one hand of efficacy of the reverse transcription step and on the other hand of synthesis initiation of the second strand. If the size of the products obtained is variable, the amplification efficacy will not be the same for all the molecules of mixture, and the measurements made during hybridization of the chips run the risk of not being reproducible. Further, for short size products, the quantity of radioactivity incorporated per molecule will be smaller than for the larger size products, introducing a potential difference during hybridization quantification.

The present invention aims to remedy the bias introduced by all of these techniques during amplification and labelling of nucleic acids of heterogeneous sizes. which bias limits the quantitative analysis of transcriptomes. It provides a process for producing a complex probe from a transcriptome that preserves each RNA representation in the final product by producing homogeneous fragments size and specific labelling and that, after a cDNA amplification step.

It is applicable to all amplification methods used to analyze a complex mixture of nucleic acids.

The products obtained are used as complex probes for studying the expression of genes, and more particularly for acquiring quantitative data on this expression.

The present invention concerns a process for producing complex probes from total RNA or messenger RNA for studying the quantitative expression of genes on DNA chips. The principal advantage over previously described methods resides on the one hand in the fact that the RNA are transcribed into cDNA of homogeneous size and that the amount of incorporated radioactivity (or any other type of labelling) is the same for all molecules present in the initial mixture and on the other hand, that the measurement is carried out on a DNA chip comprising target DNA strands characterized by a sequence representing the 3′ end of genes the transcripts of which are to be measured. The quantification carried out after hybridization on DNA chips is thus not biased by different specific activities of the synthesized cDNA.

More precisely, this method is applicable to probes produced from poly A ends of mRNA (reverse transcription of mRNA to cDNA is initiated by a poly T oligonucleotide) and to chips on which 3′ clones are deposited, i.e., cDNA (in the form of PCR products or bacterial clones) also having been obtained from mRNA 3′ ends. Actually, this process for producing cannot be applied to random initiation of the reverse transcription step of the mRNA of the probe, the cDNA of the probe having to be in the same region as the cDNA deposited on the support to allow hybridization.

More precisely, the step for homogenizing the size takes place during reverse transcription of mRNA to single strand cDNA, which step is calibrated (i.e., controlled from a kinetic viewpoint) so that the single strand cDNA are all of comparable size.

As a result, their subsequent amplification is no longer biased by the size differences between the reverse transcription products.

Thus, the present invention concerns a process for producing probes which are representative of nucleic acids population, the elements of which are to be analyzed quantitatively, characterized in that it comprises:

a) a step for calibrating experimental conditions for transcription or reverse transcription to obtain nucleic acid fragments that are substantially of the same length, said length being in the range 20 to 2000 nucleotides;

b) a step for producing a population of probe sequences from transcription or reverse transcription of the nucleic acids population, the elements of which are to be quantitatively analyzed, under conditions that have been pre-established during the preceding step, so that the probes are of homogeneous size and are representative of the 3′ portion of each element of said population;

c) an amplification step of the sequences obtained at b).

The size of the desired fragments will preferably be in the range 500 to 1500 nucleotides, and more preferably about 1000 nucleotides.

It is clear from the foregoing that the population of a probe sequence with a homogeneous size can derive from a transcription or reverse transcription step followed by amplification. The term “amplification” as used here means any technique that, from the starting sequence, whether it be RNA or DNA, can produce a large number of identical or complementary copies of said sequences. Such techniques have been described in the literature and include a number of derivatives. Examples that can be cited are PCR, RT-PCR, TMA (transcription mediated amplification), NASBA (nucleic acid sequence based amplification) and 3SR (self sustained sequence replication).

Preferably, if amplification by PCR is selected, a method that only produces non truncated fragments is preferred, preferably using the following technique:

The complex RNA mixture is reverse transcribed using the process described at a) and b) from a primer containing a poly T sequence flanked by a known sequence, or 3′ anchor, in which primers for carrying out nested PCR are chosen. The single strand cDNA obtained are 5′ ligated thanks to RNA ligase from T4 phage (T4 RNA lig) with a modified oligonucleotide with a selected sequence (5′ phosphate end for ligating with the 3′ OH end of cDNA, and 3′ NH₂ end to prevent ligation of the oligonucleotide to itself). This 5′ anchor contains sequences for a plurality of nested PCR primers compatible in PCR terms with the primers of the 3′ anchor.

The selected sequence of anchors has the following characteristics:

the anchors sequence must not have homologies with the species sequences of the transcriptome of which is being studied;

their base composition will preferably be of 60% GC to improve the quality of hybridization;

the anchor size can be from 20 to 70 nucleotides, preferably 50 nucleotides to be able to select up to 3 nested PCR primers inside them.

These PCR oligos will be chosen in pairs to account i) for the compatibility of hybridization temperatures, ii) for the absence of hairpin loops and iii) for the absence of formation of stable intra- and inter-molecular dimers.

The double strand products obtained are then all of the same size and have known anchors at their 5′ and 3′ ends; it is possible to amplify by nested PCR all the sequences present in the mixture thanks to primers selected from 5′ and 3′ anchors. This process avoids non specific priming observed, for example, when using a homopolymeric primer for anchored PCR employing tailing. This ligation technique can also be applied to linear amplification.

The probes are labeled upon transcription, reverse transcription of if appropriate, amplification of the transcripts or reverse transcripts. Labelling is carried out using any known means for labelling oligonucleotides during synthesis. Examples of the most conventional methods that can be cited are radioactivity by incorporating radioactive triphosphate nucleotides, fluorescence by incorporating fluorescent nucleotides, or incorporating nucleotides comprising a chemical modification which produces a bond with a compound that is directly or indirectly susceptible of emitting a fluorescent, phosphorescent, luminescent or colorimetric signal. A routine example today of that type of labelling is to couple the nucleotide to biotin, in which the signal is produced by coupling biotin to avidin or streptavidin carrying an enzyme, itself susceptible of transforming a substrate into a fluorescent or luminescent or colored molecule.

In the process of the invention, calibration step a) for producing transcripts or reverse transcripts of a previously selected homogeneous size consists in:

preparing a complex mixture of nucleic acids;

forming an incubation mixture comprising said mixture, a transcriptase or a reverse transcriptase, four triphosphate deoxynucleotides at least one of which is labeled and the assembly of reagents allowing enzymatic reaction;

incubating said mixture at the enzyme activation temperature;

removing aliquots during the incubation period;

analyzing the size of the reaction products for each aliquot;

selecting the incubation period that produces cDNA with the previously selected homogeneous size.

These steps will not be carried out for each probe production, but serve to establish the optimum reverse transcription condition which will be applied for all of the probes produced under similar conditions, in particular using the same enzyme.

Regarding preparation of a complex mixture of nucleic acids, in this case we prepare total RNA in a sufficient quantity for producing a kinetics for selecting the optimum incubation period for a given reverse transcript size.

Analysis of the size of the reaction products that can establish the optimum incubation period can be carried out using any means that is known to the skilled person. These include electrophoresis on denaturing gel (5).

In order to check that the calibration is correct, a “control” hybridization is carried out on a chip on which are complementary DNA of 3 RNA with different sizes added to initial RNA mixture in the same proportions: for example, RNA of 0.5, 1 and 2.5 kb are introduced in an amount of 2% (for example: 0.2 ng of each for 5 μg of total RNA). Quantification of these control RNA spots will give the same intensities for the three different sizes if the condition is correct.

Once the optimum condition has been determined, this condition is applied to a sample containing the transcriptome to be studied. It can also be applied firstly to reverse transcription of the transcriptome then to amplification of the complex mixture obtained. As indicated above, the PCR amplification products pose a real problem in that the size of the amplified product depends on the one hand of efficacy of the reverse transcription step, and on the other hand of initiating synthesis of the second strand and amplification steps. Thus, the shorter the starting matrix, the greater the tendency of the amplification product to become the major product in the final product.

This is true for all amplification techniques described in the literature, and particularly with the improved amplification technique described above consisting of flanking a known sequence with a poly T primer to obtain in 5′ and 3′ known anchors. The process of the invention, comprising a step for calibration and producing transcripts or reverse transcripts of a homogeneous size and representative of the 3′ portion of messenger RNA, enables the use of an amplification technique carried out on these transcripts or on these reverse transcripts leading to a population of amplified sequences representative of the starting sequences to be envisaged. The amplification products should also be considered to be representative of starting transcriptome since the amplification step from a matrix composed of fragments of homogeneous size no longer has this bias resulting from the fragment size difference.

Quantitative analysis of a transcriptome is advantageously carried out by simultaneous analysis of a large number of hybridizations between the probes and target sequences representative of the genes of corresponding genome. The process of the invention is particularly advantageous since it is applied to carrying out hybridizations on macro- or micro-arrays or on DNA chips.

The present invention also concerns a process for quantitative analysis of a transcriptome and is characterized in that it comprises:

a) calibrating to determine the optimum conditions for producing probes with a homogeneous size;

b) producing labeled probes constituted by a complex mixture of labeled nucleic acids representative of the transcriptome by carrying out the process described above;

c) amplifying the probes obtained at b) using any technique that is known to the skilled person such as PCR, anchored PCR, nested PCR, RT-PCR, TMA or NASBA;

d) preparing a support on which are fixed in an ordered way cDNA (targets) corresponding to the 3′ ends of mRNA of interest each representing a different gene;

e) hybridization of probes in b) or c) with the targets in d);

f) quantitatively measuring the labelling obtained for each target.

In step c) above, the improved anchored PCR method described above is preferably used, in which the poly T primer is flanked by a known anchor and cDNA ligated with a 5′ anchor using T4 RNA ligase.

In step f) above, the measured labelling reflects accurately relative quantities of the different elements of the transcriptome being studied.

Thus, the invention process for quantitative analysis of a transcriptome can overcome the different bias of existing methods cited in the introduction and which would lead to an overestimation or underestimation of the quantity of certain categories of messenger RNA in a given transcriptome. Here, the relative proportions of the different messenger RNA are the same after the reverse transcription and amplification steps.

The process of the invention enables a “photograph” of a given transcriptome to be produced and thus, if appropriate, allows it to be compared with another. This is particularly advantageous in different situations in which a physiological or pathological state of a cell is to be analyzed, or the effect of a treatment is to be analyzed.

In this process, the labeled probes of the mixture at b) or c) are of homogeneous size and contain between 20 and 2000 nucleotides, and preferably between 500 and 1500 nucleotides. An optimum length is about 1000 nucleotides. Clearly, in a process that involves an amplification step, the first transcription or reverse transcription step leads to the constitution of nucleic acids fragments of homogeneous size but not labeled. The labeled probes are then produced by amplification of this intermediate mixture.

In the process of the invention, hybridization of amplified probes with fixed targets on the chips is preferably carried out with an excess of targets over probes so that the fixed quantity to the target of the corresponding species is proportional to its relative abundance in the initial mixture. It is essential in the process of the invention that the measurements carried out after hybridization on the chip are effectively quantitative and reflect the rate of expression of the transcriptome and its variability.

In addition, and to guarantee the reliability of quantification results, it is advantageous to include control reagents.

Thus, in the process of the invention, it is advantageous to incorporate into the transcriptome at least one exogenous mRNA in a known quantity and in step d), a hybridizable target with the same RNA molecule or molecules or with the reverse transcription product.

On the one hand, it is possible to use reagents that can quantitatively calibrate the measurement. To this end, internal controls are included that consist of simultaneously carrying out hybridization of probes corresponding to the transcriptome with the targets and hybridization of an exogenous probe with a complementary target on the same chip. The use of such an external standard has been described in (3). In this article, a cytochrome C554 (or CGO3) sequence from A. thaliana which has no homology with mammalian DNA was integrated as a spot on the chip and as a probe in the transcriptome sample. Quantification of obtained signals for these positive internal controls could produce an estimation of the abundance of other RNA in the sample. These controls also allow a comparison of a plurality of independent experiments in a reliable manner, as they correct differences in labelling, washing, exposure time and any eventual loss of material on the membranes after several successive hybridizations (3). In the process of the invention, the measurement can be quantitatively calibrated by measuring in the probe the mRNA corresponding to ubiquitous genes or housekeeping genes the level of expression of which is known to be constant in all of the samples being studied.

On the other hand, it is possible to use reagents that can control efficacy of the calibration method. To this end, an internal control of validity of the measurements is made by incorporating into the RNA sample to be analyzed 0.05% to 0.2% or more of at least two exogenous RNA synthesized in vitro. They may, for example, be a 1 kb RNA from cytochrome C554 (or CG03) and two other exogenous RNA of different size, for example 0.5 kb and 2.5 kb, which can advantageously verify that the length does not influence the signal intensity when reverse transcription is carried out for a period determined by the calibration method of the present invention.

A negative control can also be carried out: clones containing polyA sequences are deposited in a regular pattern on the membrane to check that the polyT sequences introduced during synthesis of the complex probe by reverse transcription does not induce background noise.

Finally, deposition of clones containing the empty “vector” (or the absence of a deposit at certain positions) on the membrane enables the background noise to be measured and optionally allows specific signals to be deduced.

In the process of the invention, the chips can be micro- or macro-arrays. The support can be silica, glass or nylon as has been described in detail in (1) which provides the respective performances of nylon and glass supports. The chips carry 1 to 100000 targets. This clearly depends on the surface of the chip itself and on the transcriptome being studied. In certain cases, it may be advantageous to use low density membranes, the format of which can be adapted to the number of genes studied or to the quantity of available starting material.

High density membranes on a nylon support can also be developed. Said membranes can have a microarray type format (the size of a microscope slide) or a macroarray type format (about 10 to 100 cm²). These high density chips allow simultaneous analysis of several thousands of genes.

The targets can be oligonucleotides or purified cDNA fixed using methods that are well known to the skilled person, from the moment when the fraction corresponding to the 3′ portion of the mRNA of the transcriptome is accessible to hybridization.

The targets can also be bacterial clones the genome of which carries a sequence to be quantified or a complementary sequence thereof. Techniques for depositing these clones or purified sequences on the chips can be used and are known to the skilled person. Clearly, any other technique that can leave the complementary sequences of the 3′ portion of the messenger RNA accessible can be used in the process of the invention.

The present invention also concerns a kit for the quantitative study of the variability of a transcriptome, characterized in that it comprises at least:

free dNTP one of which is labeled;

a reverse transcriptase;

at least two control probes of quantitative validation, constituted by two sequences of nucleic acids with a predetermined size and each being different, which do not form part of the transcriptome or are not hybridizable with elements of the transcriptome;

a support on which are fixed in an ordered way target sequences that can be hybridized with the 3′ copies of mRNA of the transcriptome, and at least two targets that are hybridizable with the control probes.

The kit of the invention can also contain all of required reagents for carrying out the amplification.

In the kit of the invention, the targets on the support are purified cDNA. However, the targets can also be bacterial extracts the genome of which or the plasmids of which contain sequences that can be susceptible of being hybridized with the probes.

The nylon support is suitable both for depositing low density of bacterial colonies (for example 36 deposits per cm²) and for depositing PCR products at low, medium or high densities (up to 2000 deposits per cm2). It allows detection of hybridization complexes by radioactivity (the most sensitive), and is also suitable for non isotopic detection methods such as chemiluminescence (5) or colorimetry (4). Finally, as previously seen above, the performances of nylon/radioactive probe microarrays remain unequalled and are suitable for thematics in which the size of sample to be treated is reduced (biopsies, primary cell cultures).

The experiments below indicate that the calibration method leading to the production of reverse transcripts with a homogeneous size effectively lead to the production of quantitative and reproducible results as regards the rate of hybridized probes on the targets.

### KEY TO FIGURES

FIG. 1 shows an image obtained after hybridization of a membrane containing 1056 spots (each spot corresponding to a mouse cDNA clone) with a complex probe produced by applying the standard process of the invention to total mouse thymus RNA in two hours of reverse transcription.

The circled spots correspond to clones in which the level of expression is much higher when transcription is for a long period, and for which the mRNA are long.

The spots bordered by squares correspond to clones in which the level of expression is constant regardless of the transcription period and for which the mRNA are small.

FIG. 2 shows an image obtained after hybridization of a membrane containing 1056 spots with a complex probe produced by applying the process of the invention to total mouse thymus RNA with 30 minutes of reverse transcription.

FIG. 3 shows a Northern Blot carried out from total brain RNA and hybridized with a probe produced from a clone the measured level of expression of which is constant regardless of the transcription period. The observed band corresponds to a mRNA of 200 nucleotides, as indicated on the molecular weight scale.

FIG. 4 shows labeled cDNA radioactively obtained by reverse transcription (15 min, 30 min, 1 h and 2 h of RT) and migrated on a denaturing alkaline gel that can analyze the size of cDNA (molecular weight scale on the left).

### EXPERIMENTAL PROTOCOLS

1. Membrane Preparation:

Membranes were prepared using the protocols described in (2) and (3). After depositing on the membranes, bacterial clones or target DNA produced by PCR were denatured in situ then neutralized..

2. Oligonucleotide Hybridization (Vector):

Hybridization of the vector served to measure the rate of DNA in each spot in order to carry out a correction of the values obtained with complex probes. It is important to expose for a sufficient time and to correctly quantify this hybridization before that of the complex probe.

2.1 Preparation of Radiolabeled Oligonucleotide:

The oligonucleotides used depend on the sequences flanking the target cDNA, for example, for the pcDNA1 and pT7T3 plasmids:

pcDNA1: 5′gcttatcgaaattaatacgactcactatag

pT7T3pac: 5′tgtggaattgtgagcggata or

T7: taatacgactcactataggga

2.2 Labelling was Then Carried Out by Incubating the Oligonucleotides in the Presence of 1 μl of Oligo (1 μg/μl), 2 μl of 10×T4 Polynucleotide Kinase Buffer (Biolabs), 3 μl of γAT³³P (5000 Ci/mM), 1 μl of T4 Polynucleotide Kinase (10 U/ul, Biolabs), Sterile Water Up to 20 μl Final.

2.3 Precipitation (to Eliminate the Majority of Non Incorporated ATP):

The DNA was then precipitated in the presence of 1 μl of herring sperm DNA (Boehringer, 10 mg/ml), 2 μl of 3M sodium acetate, 60 μl of cold absolute ethanol (−20° C.).

The reaction mixture was kept at −80° C. for 15 minutes, centrifuged for 30 min at 4° C. then the supernatant (highly radioactive) was eliminated. The pellet was re-suspended in 100 μl of sterile water.

Counting was carried out by liquid scintillation.

2.4 Hybridization of Oligonucleotide Probe:

The hybridization/pre-hybridization buffer was constituted by 5× SSC, TX Dehnardt's, 0.5% SDS (buffer H).

2.4.1 Pre-Hybridization:

100 μg/ml (final concentration) of sonicated herring sperm DNA (Boehringer) was added to 50 ml of buffer H. The stock solution was at a concentration of 10 mg/ml and the required aliquot was denatured just before its use by heating for 10 minutes at 100° C. then cooling rapidly to 0° C. by placing the tube in ice for 10 minutes.

The filters were pre-hybridized at 42° C. for a minimum of 4 hours (maximum 12 h) in buffer H (50 ml of buffer and a maximum of 4 filters per dish or 10 ml in the tubes).

2.4.2 Hybridization:

The filters were hybridized in 50 ml of buffer H then withdrawn. A hot/cold probe mixture was added to the buffer. The filters were then replaced in the dish one by one. Hybridization was carried out at 42° C. for at least 12 hours and with stirring in a moist atmosphere to prevent any potential evaporation.

The cold/hot probe mixture was constituted by 10 μg of cold oligo vector, 100000 to 200000 cpm/ml of labeled oligo (i.e, 5 to 10 million per 50 ml).

2.4.3 Washes and De-Hybridization:

The washes were carried out with 1 liter of 2× SSC 0.1% SDS buffer, for 10 minutes, at ambient temperature then for 5 minutes at 42° C., changing the buffer once.

The membranes were exposed to a phosphorous screen which was then read using a Fuji Bas 1500 type apparatus.

2.4.4. De-Hybridization:

De-hybridization was carried out in 0.1× SSC/0.1% SDS for 3 hours at 68° C., changing the buffer once.

3. Labelling Complex Probes Using 5 μg of Total RNA:

3.1 Preparation of Radiolabeled Probe:

The complex probe was prepared from total RNA extracted from the sample of interest (tissue, cell culture, . . . ). The first step was an annealing step (in order to RNA lose their secondary structure and to saturate the polyA tails with oligo dT). A large excess of oligo dT was used so that RT transcription began just after the start of the polyA tail. The conditions were as follows: 5 μg of RNA and 0.2 ng of CG03 (cytochrome) control mRNA, also 0.2 ng of each other exogenous RNA acting to control quantitative validation, and 8 μg of dT25 were added to 13 μl of sterile water and incubated for 8 minutes at 70° C., then for 30 minutes at 70° C. to 42° C.

3.2 Reverse Transcription (to Simultaneously Synthesize and Label Single Strand DNA Corresponding to About 100 ng of mRNA Present in 5 μg of Total RNA).

This was carried out under the following conditions: 1 μl of RNAsin (Ribonuclease inhibitor, Promega, Ref. N2511, 40 U/μl ), 6 μl of first strand 5× (BRL) buffer, 2 μl of 0.1 M DTT, 0.6 μl of 20 mM dATG (20 mM each), 0.6 μl of 120 μM dCTP, 3 μl of 10 μCi/μl (a³³P)dCTP (>3000 Ci/mM), 1 μl of reverse. transcriptase (SUPERSCRIPT RNase H free RT, BRL, 200 U/μl), and sterile water up to 30 μl were added to the sample at 42° C.

a) Conventional Protocol:

incubate 1 hour at 42° C. (incubator);

add 1 μl of reverse transcriptase;

incubate further 1 hour at 42° C. (incubator).

b) Calibration Protocol:

The RT reaction was stopped after 15 minutes, 30 minutes, 1 hour or 2 hours (i.e. 4 reactions with non precious RNA). The reaction was stopped by passing into ice then alkaline lysis of RNA or using any other method known to the skilled person: for example RNase H or adding ddNTP.

These four “test” probes were hybridized on arrays containing at least the three complementary targets of 3 exogenous RNA with different sizes (500/1000/2500) introduced in the same proportions into the RNA of the initial mixture.

That of the four conditions that could produce signals of the same intensity for the control spots was defined as the optimum condition for the remaining experiments using: the same RTase, the same experimental conditions throughout the protocol.

The reaction mixture was then neutralized by adding 10 μl of 1M TRIS, 3 μl of 2N HCl and sterile water, up to-150 μl.

The complex probes were purified on a 1 ml Sephadex G50 column (Pharmacia).

c) PCR Amplification Protocol:

Anchors were added at the 3′ and 5′ of the cDNA obtained. The 3′ anchor was an oligo dT containing in its 5′ portion the promoter sequence for T7 RNA polymerase and a complementary sequence of the SP6 promoter at the 5′ anchor.

The first anchor was added during reverse transcription carried out under the conditions defined at b), and the second anchor was added by ligation with T4 RNA ligase, to the 3′ end of the synthesized cDNA.

PCR amplification was carried out with a pair of complementary primers to the 3′ and 5′ anchors, namely T7 and SP6.

Labelling was carried out during or after the PCR.

4. Hybridization of the Complex Probe:

The hybridization conditions were the same as those described in 2.4 above, with the following modifications:

the pre-hybridization step was carried out between 65° C. and 68° C. for a minimum of 6 hours in buffer H;

the hybridization step was carried out between 65° C. and 68° C. for 48 hours.

Whole of the labeled probe had to be added so as not to modify the concentration of RNA species and render comparison with the other experiments difficult;

washes were carried out in 1 liter (for 4 filters) of 0.1× SSC, 0.1% SDS solution at 68° C. for 3 hours, changing the washing solution once.

(The washing solution was pre-heated at 68° C.).

The membranes were exposed to a phosphorus screen which was then read using a Fuji Bas 1500 type apparatus.

De-hybridization was carried out in 0.1% SDS/1 mM EDTA at 80° C. for 2 hour 30 minutes (1 liter for 4 filters).

### EXAMPLE 1

Size of cDNA Obtained After 4 Different Reverse Transcription Periods:

The starting RNA were total mouse brain RNA.

The reverse transcription reaction (RT) was carried out using the protocol described in the publications above and in (2) and (3).

Four reactions were carried out in parallel:

reaction of 2 hours (“conventional” protocol, 1 hour RT, adding 1 μl of enzyme and another 1 hour of RT);

1 hour of RT;

30 minutes of RT;

15 minutes of RT.

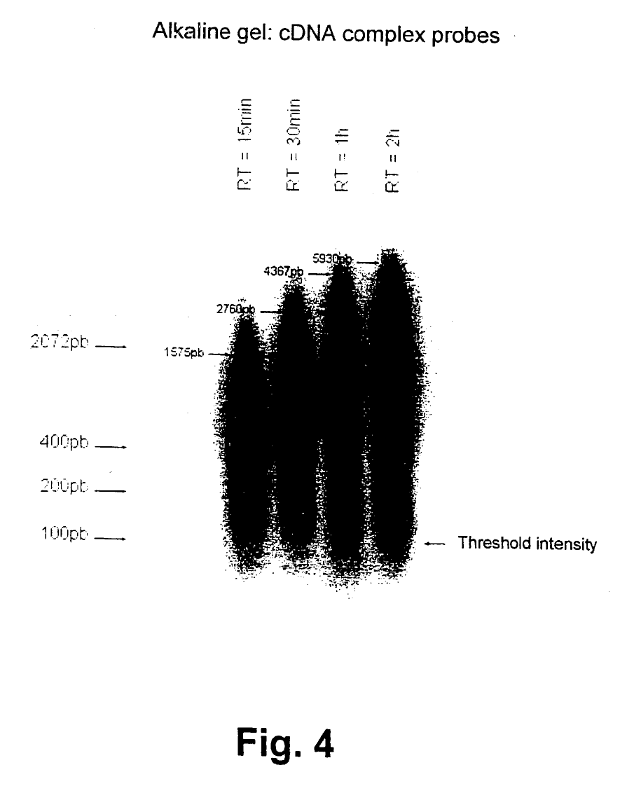

The RT products (into which phosphorus-32 radioactive labeled nucleotides had been incorporated) were then deposited on a denaturing alkaline gel (5) with a molecular weight marker, then visualized by autoradiography. The results obtained are shown in FIG. 4.

The approximate sizes observed were respectively:

RT 2 hours: 100<cDNA<5930 nucleotides

RT 1 hour: 100<cDNA<4367 nucleotides;

RT 30 min: 100<cDNA<2760 nucleotides;

RT 15 min: 100<cDNA<1575 nucleotides.

### EXAMPLE 2

Quantitative Analysis of Transcriptome:

The cDNA reverse transcribed from mouse thymus RNA were hybridized on chips carrying cDNA from genes expressed in the mouse and carried by bacterial plasmids.

After carrying out the calibration method of the invention, the reverse transcription reaction period was fixed at 30 minutes.

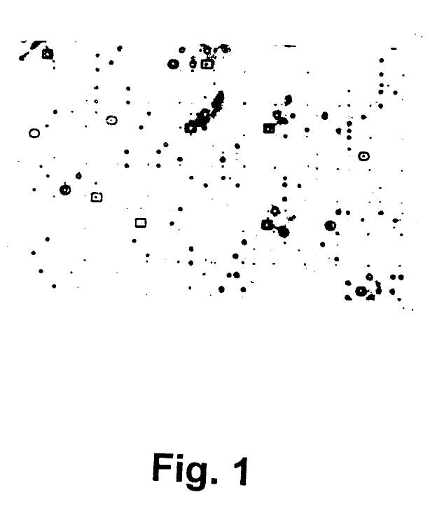

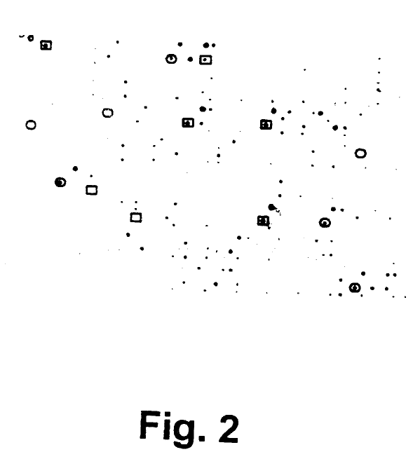

FIG. 1 shows the intensities of spots obtained after the conventional reverse transcription period (2 hours, FIG. 1), and during the selected period of 30 minutes, deduced from the previous calibration operation (FIG. 2).

The intensity of spots obtained was compared under these two experimental conditions and the results are shown in the following table:

| | | Filter n° 7, 2 | Filter no 10, 30 | |
|---|---|---|---|---|
| | | h | min | |
| | Clones | RA* | RA* | 2 h/30 min |
| A) | MTB.CO7.023 | 154.13 | 14.09 | 10.94 |
| | MTB.O02.020 | 113.56 | 13.51 | 8.40 |
| | MTB.K10.017 | 195.45 | 23.57 | 8.29 |
| | MTB.E15.014 | 112.70 | 15.08 | 7.48 |
| | MTB.013.002 | 4.11 | 0.32 | 12.81 |
| | MTB.G11.025 | 6.83 | 0.96 | 7.14 |
| | MTB.A10.005 | 4.97 | 0.77 | 6.44 |
| B) | MTB.L05.018 | 209.21 | 100.53 | 2.08 |
| | MTB.N23.020 | 281.10 | 145.92 | 1.93 |
| | MTB.F04.013 | 212.05 | 111.67 | 1.90 |
| | MTB.A22.001 | 119.33 | 77.62 | 1.54 |
| | MTB.L08.017 | 4.32 | 4.47 | 0.97 |
| | MTB.E05.028 | 1.88 | 2.64 | 0.71 |
| | MTB.F17.006 | 2.16 | 3.68 | 0.59 |

These values were obtained after quantification using Biolmage software (Fuji) of the images in FIGS. 1 and 2. The values given are relative abundances (RA: abundance of mRNA in each clone in the transcriptome used to produce the complex probe). The first column informs about the clone name; the second and third columns show the relative abundances of these clones for filters n° 7 (column 2) and n° 10 (column 3), and the last column gives the ratio of the 2 hours/30 minute measurements for each clone.

The first seven ringed clones in FIGS. 1 and 2 had expression measurements that were substantially higher when the probe was produced over two hours. The other clones bordered by squares in FIGS. 1 and 2 and corresponding to group B in the tables had constant expression measurements regardless of the transcription period.

It can be seen by observing the values of the table that there is a much greater stability in the ratio in the serie of spots B than in the serie of spots A. Actually, in spots A, the radioactivity measured for each spot was much higher after two hours than after 30 minutes of reverse transcription. This corresponded to long RNA as shown in the Northern Blot experiments carried out subsequently.

In contrast, the stability of the two hours/30 minutes ratio in series B was indicative of short mRNA hybridization.

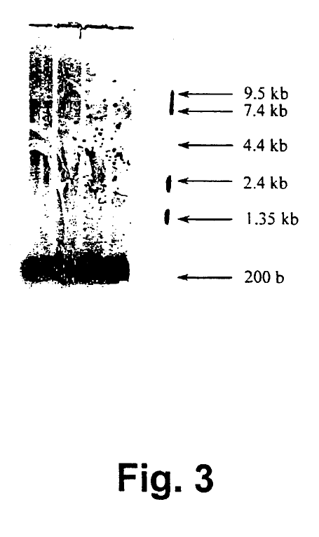

This was verified by Northern Blot hybridization which effectively shows that the size of the RNA molecules in question was 200 nucleotides, as shown in FIG. 3.

It can be concluded from this experiment that when the RNA molecules are short, no bias is imported into the quantitative measurement of the quantity of RNA present. In contrast, when the RNA are long, the quantity of labelling measured was variable and thus, one spot cannot be compared with another.

This experiment validates the fact that comparison of the intensities of labelling obtained from one spot to another is reliable for short reverse transcripts.

### REFERENCES

(1) Granjeaud S., Bertucci F. and Jordan B. R. Expression profiling: DNA arrays in many guises. BioEssays, (1999) 21: 781-790

(2) Bertucci F., Van Hulst S., Bernard K., Loriod B., Granjeaud S., Tagett R., Starkey M., Nguyen C., Jordan B. Birnbaum D. Expression scanning of an array of growth control genes in human tumor cell lines. Oncogene, 1999 July, 18(26): 3905-3912

(3) BeRNArd K. et al., Nucl. Acids Research (1996) 24(8): 1435-42

(4) Chen et al., Genomics (1998) 51: 313-324

(5) Rajeevan M. S., Dimulescu J. M., Unger F. R., Vernon S. D. Chemiluminescent analysis of gene expression on high-density filter arrays. J. Histochem Cytochem, 1999 March, 47(3): 337-342

1. A process for producing probes that are representative of a population of nucleic acids the elements of which are to be analysed quantitatively, characterized in that it comprises:
a) a step for calibrating the experimental conditions for transcription or reverse transcription to obtain nucleic acid fragments with a homogeneous size;
b) a step for producing a population of probe sequences from transcription or reverse transcription of the population of nucleic acids the elements of which are to be quantitatively analysed, under incubation time conditions that have been preestablished during the previous step, so that the probes are of homogeneous size and are representative of the 3′ portion of each element of said population;
c) a step for amplification of the sequences obtained at b).

2. The process according to claim 1, in which calibration step a) to obtain transcripts or reverse transcripts of a previously selected homogeneous size consists in:
a preparing a reference complex mixture of nucleic acids;
forming an incubation mixture comprising said mixture, a transcriptase or a reverse transcriptase, four triphosphate deoxynucteotides at least one of which is labeled and the whole of reagents allowing enzymatic reaction;
incubating said mixture at the enzyme activation temperature;
removing aliquots during the incubation period;
analyzing the size of the reaction products;
selecting the incubation period that produces cDNA with the previously selected homogeneous size.

3. The process according to claim 2, in which the initial mixture is a mixture of RNA and the enzyme is a reverse transcriptase.

4. The process according to claim 1, in which the probes with homogeneous size are amplified notably by PCR, RT-PCR, TMA, NASBA, 3SR, nested PCR or anchored PCR.

5. The process according to claim 4, in which the anchored PCR is improved by:
a) adding a poly T primer to the 3′ end, which is flanked by an oligonucleotide with a known sequence in which primers that can carry out nested PCR have been selected;
b) 5′ ligation with RNA ligase of the T4 phage of an oligonucleotide with a known sequence and containing sequences for a plurality of primers for nested PCR compatible with the primers for the 3′ anchor;
said sequences at a) and b) not having homologies with the sequences of the species the transcriptome of which is being studied.

6. The process according to claim 1, characterized in that the population of nucleic acids the elements of which are to be quantititatively analyzed is a population of cellular messenger RNA or transcriptome in which the expression variability between different cellular populations is to be analyzed, and reverse transcription is initiated at the 3′ end by a poly T oligonucleotide.

7. The process according to claim 1, in which the quantitative analysis is hybridization on a microarray carrying oligonucleotide sequences that can hybridize with the transcripts or reverse transcripts.

8. The process for quantitative analysis of a transcriptome, characterized in that it comprises:
a) calibration employing the method defined in claim 2;
b) producing labeled probes with homogeneous size constituted by a complex mixture of labeled nucleic acids representative of transcriptome and obtainable by the process defined in claim 1;
c) amplifying the products obtained at b) in particular by PCR, anchored PCR, nested PCR, RT-PCR, TMA or NASBA;
d) preparing a support on which cDNA (targets) corresponding to the 3′ ends of the mRNA of interest each representing a different gene are orderly fixed in an array;
e) hybridization of the probes obtained at b) or c) with the targets in d);
f) quantitatively measuring of the labelling obtained for each target.

9. The process according to claim 8, in which the labeled probes of the mixture obtained at c) are homogeneous in size, in the range 500 to 1500 nucleotides.

10. The process according to claim 8, in which the amplification step at c) is an anchored PCR amplification comprising:
a) adding a poly T primer to the 3′ end which is flanked by an oligonucleotide with a known sequence in which primers that can carry out nested PCR have been selected;
b) 5′ ligation with RNA ligase of the T4 phage of an oligonucleotide with a known sequence and containing sequences for a plurality of primers for nested PCR compatible with the primers for the 3′ anchor.

11. The process according to claim 8, in which hybridization at e) is carried out with an excess of fixed targets compared with the hybridized probes so that the quantity fixed to the target of the corresponding species is proportional to its relative abundance in the initial mixture.

12. The process according to claim 8, in which at least one exogenous mRNA is incorporated into the transcriptome in a known quantity and in step d) a target is incorporated that is hybridizable with the same RNA or RNA molecules or with the reverse transcription product.

13. The process according to claim 8, in which at least one ubiquitous RNA (or household RNA) is measured in the transcriptome, and wherein a target that is hybridisable with the same RNA or RNA molecules or with the product of its reverse transcription has been incorporated into step d).

14. The process according to claim 8, in which an internal control of quantitative validation is introduced by incorporating into the complex mixture formed at b) or c) at least two fragments of nucleic acid of different sizes and with a size that is different from that selected for the probes of the complex mixture, and a sequence that is hybridisable thereto is incorporated onto the support.

15. The process according to claim 14, in which when the probe size is about 1000 nucleotides, the sizes of the nucleic acid fragments are respectively about 500 nucleotides and 1500 nucleotides.

16. The process according to claim 8, in which the support in d) carries 1 to 100000 targets.

17. The process according to claim 8, in which the fixed targets in d) are purified cDNA.

18. The process according to claim 8, in which the targets are bacterial clones the genome of which carries the sequence for which quantification is desired.

19. A kit for the quantitative study of the variability of a transcriptome, characterized in that it comprises at least:
free dNTP, one of which is labeled;
a reverse transcriptase;
at least two quantitative validation controls constituted by two sequences of nucleic acids with a predetermined size and each being different, which do not form part of the transcriptome or are not hybridizable with the elements of the transcriptome;
a support on which are fixed in an ordered way target sequences that can be hybridized with the 3′ copies of the mRNA of transcriptome, and at least two targets that are hybridizable with the external standards.
